(19) United States
(12) Patent Application Publication
(10) Pub. No.: US 2025/0221440 A1
(43) Pub. Date: Jul. 10, 2025

(54) A METHOD FOR PROVIDING AN AMORPHOUS HMO PRODUCT BY DRYING

(71) Applicant: DSM IP Assets B.V., HEERLEN (NL)

(72) Inventors: Carsten Ydergaard LARSEN, Horsholm (DK); Pierre CHASSAGNE, Horsholm (DK); Markus NOWOTNY, Kaiseraugst (CH); Josef Theodor HÖRMANSPERGER, Kaiseraugst (CH); Arthur Maurits Christiaan JANSE, ECHT (NL); Patricia TANNER, Kaiseraugst (CH)

(21) Appl. No.: 18/701,863

(22) PCT Filed: Oct. 18, 2023
(86) PCT No.: PCT/EP2022/078925

§ 371 Date: Apr. 16, 2024

(30) Foreign Application Priority Data
Oct. 18, 2021 (DK) PA 2021 00995
Oct. 18, 2021 (DK) PA 2021 00996

(51) Int. Cl. A23L 33/00; C07H 1/08

(52) U.S. Cl. CPC A23L 33/40; C07H 1/08

(57) ABSTRACT

A method for providing a solid amorphous HMO product, said method including drying an aqueous composition of said HMO. Solid amorphous HMO product is obtained via said method, as well as solid amorphous HMO product per se.

### TECHNICAL FIELD

A method for providing a solid amorphous HMO product is provided.

### BACKGROUND

Human milk oligosaccharides (HMOs) are a heterogeneous mixture of soluble glycans found in human milk. They are the third most abundant solid component after lactose and lipids in human milk and are present in concentrations of 5-25 g/l (Bode: Human milk oligosaccharides and their beneficial effects, in: Handbook of dietary and nutritional aspects of human breast milk (Zibadi et al., eds.), pp. 515-31, Wageningen Academic Publishers (2013)).

Human milk oligosaccharides (HMOs) have become of great interest in the past few years due to their important functions in human development. To date, the structures of around 130 HMOs have been determined (see Urashima et al.: Milk Oligosaccharides, Nova Biomedical Books, New York, 2011, ISBN: 978-1-61122-831-1; Chen Adv. Carbohydr. Chem. Biochem. 72, 113 (2015)), and considerably more are probably present in human milk.

Low cost ways have been sought for making industrial quantities of as many as possible of the HMOs, so that their uses in nutritional and therapeutic formulations for infants, as well as possibly children and adults, could be discovered, developed and exploited by researchers worldwide. A few HMOs have recently been chemically synthesized in high yields, while other methods have used fermentation techniques using cultured microorganisms.

Whatever ways are used to produce HMOs, the final step is to obtain the purified HMO in solid form before packaging or formulation (e.g. using HMOs to make infant formula). Certain HMOs may be crystallized, while others are available only as amorphous solid. Nevertheless, HMOs are often isolated as amorphous solid, usually from their aqueous solution by removing water in a drying step.

One of the methods of drying is direct (or convective) drying where hot process gas (air, inert gas or mixture thereof) is applied that carries away the vapor as humidity. A typical example of direct drying which is often used to obtain an amorphous HMO powder is spray-drying (see e.g. WO 2015/106943, WO 2019/160922), where the liquid solution of the HMO to be dried is atomized into tiny droplets and let the droplets directly contact and mix with the hot gas of the drying medium to evaporate the water, and then collecting the dried product by gas-solid separation. Although frequently used, one may face several disadvantages: the thermal efficiency is not high and the energy consumption is large, generally two levels of dust removal are required, and high demand of drying gas/air. Another direct drying method is belt-drying which may provide dried crystalline substance instead of dried amorphous powder (WO 2021/186258).

In indirect (or contact) drying, heating is applied via a solid surface/wall to dry an aqueous concentrated solution of HMOs (see e.g. WO 2021/094459, WO 2021/155157). Again, this method can easily provide crystalline end product.

It is an object of the present invention to provide a method for providing solid amorphous HMO products, preferably by means of indirect drying, that is versatile, gentle, economical, easily up-scalable, easy to operate and suitable for industrial use.

### SUMMARY

In a first aspect the present invention relates to a method for drying an aqueous composition of one or more human milk oligosaccharides (HMOs) and thus providing a solid amorphous powder product comprising said one or more HMOs, the method comprising:
a) providing a dry amorphous powder comprising said one or more HMOs in a dryer suitable to agitate the dry amorphous powder mechanically, to obtain a dry agitated amorphous powder, preferably the dryer is designed to generate maximal possible dryer wall contact of the powder by mechanical mixing,
b) feeding the aqueous composition of the one or more HMOs in, on or to the dry agitated amorphous powder to obtain a wet agitated mixture of the amorphous powder,
c) removing water from the wet agitated mixture of the amorphous powder at elevated temperature and/or under vacuum while the aqueous solution of the one or more HMOs is continuously or sequentially added to the wet agitated mixture of the amorphous powder.

In a second aspect, the present invention relates to a dried solid amorphous HMO product comprising one or more HMOs obtained or obtainable by the method described according to the first aspect of the invention.

A food product, in particular a nutritional formulation, e.g. an infant nutritional formulation, is also provided, which comprises the dried solid amorphous HMO product as described herein.

Further features of the invention are evident from the following description, numbered aspects, examples and claims.

### DETAILED DISCLOSURE

The present inventors elaborated an especially advantageous drying process to dry an aqueous composition comprising one or more HMOs, e.g. an aqueous solution or suspension, and thereby providing a dried solid amorphous HMO powder. The aqueous composition of one or more HMOs is dried in an indirect way wherein the heat emanates from the wall of the dryer and is conducted primarily through a mass of a dry HMO powder prefilled in the dryer and-during the drying process-together with the already dried mass of the HMO powder. Consequently, the aqueous composition is dried on or in a previously dried powder. The efficacy of the drying process may be enhanced by application of vacuum.

Accordingly, a first aspect of the invention relates to a method for drying an aqueous composition comprising an HMO and thereby to provide a dried amorphous powder of HMO, the method comprises:
a) providing a dry amorphous powder comprising said HMO in a dryer suitable to agitate the dry amorphous powder mechanically, to obtain a dry agitated amorphous powder, preferably the dryer is designed to generate maximal possible dryer wall contact of the powder by mechanical mixing,
b) feeding the aqueous composition of the HMO in, on or to the dry agitated amorphous powder to obtain a wet agitated mixture of the amorphous powder,
c) removing water from the wet agitated mixture of the amorphous powder at elevated temperature and/or under vacuum while the aqueous composition of the HMO is continuously or sequentially added to the wet agitated mixture of the amorphous powder.

The drying method disclosed herein possesses several advantages compared to the prior art direct drying processes (e.g. spray-drying) or contact drying processes (e.g. drum or roller drying). Most importantly, the aqueous composition of an HMO to be dried, preferably an aqueous solution, is added, preferably sprayed, in, on or to the dry amorphous powder of HMO prefilled in the dryer and not the heated wall of the dryer directly. In this regard, the drying process is delicate, no product layer is formed on the wall of the dryer and substantial degradation/browning of the HMO can be avoided. Further, the method provides a dried amorphous powder consistently, as opposed to other contact drying methods like drum-drying. In addition, as the method is operated in a closed equipment, it meets the high hygienic standards required for the production of foods or food additives, especially in the manufacture of infant formula. Moreover, no metal abrasion takes place (cp with drum-drying), and no handling of process gas/air is necessary (cp with spray-drying) that lowers the overall carbon footprint of the method. Due to the efficient heat transfer, the mixer-dryer can be compact. The drying method according to the invention removes water efficiently in a gentle way, which makes the method especially suitable to dry heat sensitive substances. Thanks to the delicate drying process, the dried material does not stick to the wall of the dryer and the dryer is easy to discharge and clean.

The method described herein uses an aqueous composition comprising the one or more HMOs. The aqueous composition may be an aqueous solution or an aqueous suspension of one or more HMOs, preferably an aqueous solution.

The aqueous composition suitably comprises one or more HMOs, such as e.g. two or more HMOs, or three or more HMOs, or four or more HMOs. The aqueous composition may comprise even 5, 6 or more HMOs. Advantageously, the HMO or HMOs are identical to that/those contained in the prefilled dry amorphous HMO powder.

The aqueous composition may include additional components such as e.g. salts, pH regulating agents or solubilising agents. The pH of the aqueous composition is suitably between 3-7, preferably between 4-6.

The one or more HMOs in the prefilled dry amorphous powder and in the aqueous composition may contain additional non-HMO carbohydrates, such as lactose, lactulose, monosaccharides (e.g. glucose, galactose, fucose, sialic acid). The overall amount of the non-HMO carbohydrates advantageously does not exceed 10 wt % (calculated on water-free solids), preferably 8 wt %. The overall amount of the non-HMO carbohydrates excluding lactose advantageously does not exceed 2 wt % (calculated on water-free solids). Consequently, the HMO content of the obtainable powder by the present method will be the same as that of the prefilled dry amorphous powder and the aqueous composition.

In the method according to the present invention, the drying step suitably takes place to a moisture content in the solid HMO product of below 10%, preferably below 8%, more preferably below 6%, for example below 4% down to 1-3%.

The human milk oligosaccharides referred to in the present technology are synthetic, i.e. produced by chemical or biochemical processes in vitro or in vivo. The synthetic HMOs used in the present methods and products may be selected from one or more of LNT, LNnT, 2′-FL, 3-FL, DFL, LNFP I, LNFP II, LNFP III, LNFP V, LNFP VI, 3′-SL, 6′-SL, FSL, LST a, LST b, LST c, and DS-LNT, preferably LNT, LNnT, 2′-FL, 3-FL, DFL, LNFP I, 3′-SL and 6′-SL.

The synthetic HMOs may be neutral or acidic (sialylated). The term “neutral human milk oligosaccharide” means a non-sialylated (therefore neutral) complex carbohydrate found in human breast milk (Urashima et al.: Milk oligosaccharides, Nova Biomedical Books, 2011; Chen Adv. Carbohydr. Chem. Biochem. 72, 113 (2015)) comprising a core structure being a lactose unit at the reducing end that is a) substituted with one or two α-L-fucopyranosyl moieties, b) substituted with a galactosyl residue, or c) elongated, via its 3′-OH group, by an N-acetylglucosamine, a lacto-N-biose (Galβ1-3GlcNAc) or an N-acetyllactosamine (Galβ1-4GlcNAc) moiety. The N-acetyllactosamine containing derivatives can be further substituted with N-acetyllactosamine and/or lacto-N-biose (lacto-N-biose is always a non-reducing terminal). The N-acetyllactosamine and the lacto-N-biose containing derivatives can optionally be substituted by one or more α-L-fucopyranosyl moieties.

Examples of neutral trisaccharide HMOs include 2′-O-fucosyllactose (2′-FL, Fucα1-2Galβ1-4Glc), 3-O-fucosyllactose (3-FL, Galβ1-4(Fucα1-3)Glc) or lacto-N-triose II (GlcNAcβ1-3Galβ1-4Glc); examples of neutral tetrasaccharide HMOs include 2′,3-di-O-fucosyllactose (DFL, Fucα1-2Galβ1-4(Fucα1-3)Glc), lacto-N-tetraose (LNT, Galβ1-3GlcNAcβ1-3Galβ1-4Glc) or lacto-N-neotetraose (LNnT, Galβ1-4GlcNAcβ1-3Galβ1-4Glc); examples of neutral pentasaccharide HMOs include lacto-N-fucopentaose I (LNFP I, Fucα1-2Galβ1-3GlcNAcβ1-3Galβ1-4Glc), lacto-N-fucopentaose II (LNFP II, Galβ1-3(Fucα1-4)GlcNAcβ1-3Galβ1-4Glc), lacto-N-fucopentaose III (LNFP III, Galβ1-4(Fucα1-3)GlcNAcβ1-3Galβ1-4Glc), lacto-N-fucopentaose V (LNFP V, Galβ1-3GlcNAcβ1-3Galβ1-4(Fucα1-3)Glc), lacto-N-fucopentaose VI (LNFP VI, Galβ1-4GlcNAcβ1-3Galβ1-4(Fucα1-3)Glc); examples of neutral hexasaccharide HMOs include lacto-N-difucohexaose I (LNDFH I, Fucα1-2Galβ1-3(Fucα1-4)GlcNAcβ1-3Galβ1-4Glc), lacto-N-difucohexaose II (LNDFH II, Galβ1-3(Fucα1-4)GlcNAcβ1-3Galβ1-4(Fucα1-3)Glc), lacto-N-difucohexaose III (LNDFH III, Galβ1-4(Fucα1-3)GlcNAcβ1-3Galβ1-4(Fucα1-3)Glc), lacto-N-hexaose (LNH, Galβ1-3GlcNAcβ1-3(Galβ1-4GlcNAcβ1-6)Galβ1-4Glc), para-lacto-N-hexaose (pLNH, Galβ1-3GlcNAcβ1-3Galβ1-4GlcNAcβ1-3Galβ1-4Glc), lacto-N-neohexaose (LNnH, Galβ1-4GlcNAcβ1-3(Galβ1-4GlcNAcβ1-6)Galβ1-4Glc) or para-lacto-N-neohexaose (pLNnH, Galβ1-4GlcNAcβ1-3Galβ1-4GlcNAcβ1-3Galβ1-4Glc).

The term “sialylated human milk oligosaccharide” means a sialylated complex carbohydrate found in human breast milk (Urashima et al.: Milk oligosaccharides, Nova Biomedical Books, 2011; Chen Adv. Carbohydr. Chem. Biochem. 72, 113 (2015)) comprising a core structure being a lactose unit at the reducing end that can be a) substituted with an α-N-acetyl-neuraminyl (sialyl) moiety or b) elongated by one or more β-N-acetyl-lactosaminyl and/or one or more β-lacto-N-biosyl units, and which core structure is substituted by an α-N-acetyl-neuraminyl (sialyl) moiety and optionally can be substituted by an α L-fucopyranosyl moiety. In this regard, the acidic HMOs have at least one sialyl residue in their structure.

Examples of acidic HMOs include 3′-sialyllactose (3′-SL), 6′-sialyllactose (6′-SL), 3-fucosyl-3′-sialyllactose (FSL), LST a, fucosyl-LST a (FLST a), LST b, fucosyl-LST b (FLST b), LST c, fucosyl-LST c (FLST c), sialyl-LNH (SLNH), sialyl-lacto-N-hexaose (SLNH), sialyl-lacto-N-neohexaose I (SLNH-I), sialyl-lacto-N-neohexaose II (SLNH-II) and disialyl-lacto-N-tetraose (DS-LNT).

Different mixtures of HMOs produced enzymatically are described in earlier patent applications WO 2016/199071, WO 2017/221208, WO 2017/103850, WO 2016/157108, WO 2016/063262, which are incorporated by reference.

Any artificial blends of HMOs synthesised by any available methods are also included in the scope of the present invention.

In step a) of the present invention, a dry amorphous powder comprising one or more HMOs is provided in a dryer suitable to agitate the dry amorphous powder mechanically, e.g. by a rotor. The dry amorphous powder suitably has a moisture content that is desired to be achieved at the end of the present drying process. The dry amorphous powder may be a product obtained after a drying process according to the present invention, a spray-dried amorphous powder, a freeze-dried amorphous powder or an amorphous powder dried by any other methods that provide the substance in amorphous form. The dryer is suitably a mixer dryer or a blender dryer, that is equipped with a tool with which an effective mechanical agitation/mixing/stirring of the dry amorphous powder, as well as the wet mixture of the amorphous powder in step b) of the method, can be conducted, e.g. shovel, propeller, turbine, paddle, spiral mixing blade, etc. Preferably, the mixer dryer is designed so that it is able to generate a maximal possible dryer wall contact of the powder by mechanical mixing. Accordingly, the term “dry agitated amorphous powder” means that the dry amorphous powder comprising one or more HMOs placed in the dryer is well mixed/agitated.

In step b) of the method according to the present invention, an aqueous composition, preferably a solution, of the HMO is added in, on or to the dry agitated amorphous powder of HMO to obtain a wet agitated mixture of the amorphous powder. Preferably, the HMO or HMOs comprised in the aqueous composition is/are identical to that/those in the dry agitated amorphous powder.

The aqueous composition may be an aqueous solution or an aqueous suspension of one or more HMOs, preferably an aqueous solution. The HMO content of the aqueous composition, and whether a solution or slurry/suspension is formed, depends the solubility of the HMO in question. Preferably, the aqueous composition does not contain a solvent different than water. In certain embodiments, the aqueous composition may contain one or more solvents different than water; this solvent may be a water-miscible solvent or a solvent that is partially miscible with water, typically those can be used in the production and/or purification of an HMO in a chemical way, or in the crystallization of the HMO or one of its precursors during the manufacture of the HMO, such as an C₁-C₄ alcohol, acetone, acetic acid, etc. In the aqueous composition of the HMO, the combined concentration of the solvents different than water is not more than 5 w/w %, preferably not more than 4 w/w %, more preferably not more than 3 w/w %, even more preferably not more than 2 w/w %, advantageously not more than 1 w/w %. Although the concentration of the HMO in the aqueous composition is not an essential factor, in order to enhance the efficacy and the feasibility of the method it is preferred that the minimum concentration of the HMO is at least 80%, preferably at least 90%, of its solubility concerning water (or optionally concerning the water-solvent mixture). Preferably, the process is run on a saturated solution or with a concentration below saturation, it but can also be imagined to be run on a supersaturated metastable solution or an aqueous slurry.

The addition of the aqueous composition of an HMO in, on or to the dry agitated amorphous powder of HMO results, under continuous agitation/mixing, in the formation of a wet agitated mixture of the amorphous powder. The aqueous composition shall be carefully added to the dry agitated amorphous powder so that no local deliquescence takes place and the aqueous composition is immediately taken up or encapsulated by the dry agitated powder. Due to the constant agitation, the water content of the aqueous composition is evenly distributed throughout the dry agitated amorphous powder resulting in a wet agitated powder, the resulting powder will be still well agitable but its water content/humidity will be higher. Preferably, the moisture/water content of the wet agitated powder is not more than 15%, for example not more than 12%, preferably not more than 10%, for example around 6-8% or 4-6%. Advantageously, the aqueous composition is added in the form of tiny droplets, e.g. in the form of a spray or sprinkle. As a consequence, sticky phases, balling or product sticking or forming crusts on the wall of the mixer dryer is largely avoided.

Preferably in step b), the aqueous composition is an aqueous solution of the HMO. In this case, the aqueous solution is sprayed in, on or to the prefilled agitated dry powder through an atomizing nozzle with the aid of compressed air. Alternatively, the aqueous solution is added through a standard liquid addition lance, which does not require pressurized air to assist the addition, therefore more beneficial if at least steps b) and c) are operated under vacuum. Whichever type of the nozzle is used, it is installed in the mixer so that the liquid solution is sprayed or sprinkled mostly, preferably entirely, in, on or to the prefilled dry agitated powder in step b) and the wet agitated powder mixture in step c).

In step c), water is removed from the wet agitated powder so that the wall of the mixer is heated while the wet powder is agitated. The skilled person is able to set or adjust the wall temperature to obtain the desired moisture content of the dried amorphous HMO product. The heat from the wall, due to the effective agitation, is then conducted evenly to the mass of the dry powder and eventually to the wet powder. For an effective water removal at atmospheric pressure, the temperature of the wet powder is kept at around 60-90° C., preferably 70-80° C., which requires that the wall temperature is set to around 80 to 140° C., more preferably to around 90-120° C. The water removed from the wet agitated powder leaves the equipment in the form of vapour via an outlet.

The present method encompasses operation under vacuum, provided the mixer dryer is suitable for that. Performing the method at reduced pressures, it can advantageously avoid issues relating to high temperatures. Vacuum mixer dryers then typically operate at 20-100 mbars of pressure, e.g. 20-60 mbars, allowing the water to be evaporated at much lower temperature, therefore exposing the product to lower temperatures (40-80° C.). Preferably, the process is performed under such vacuum, keeping the product temperature at 60-80° C. Besides operating at lower temperatures, vacuum mixer dryers are also more sanitary (as they operate within a closed environment) and thus more suitable for infant food production.

While the water is removed from the wet agitated powder under the conditions disclosed above, an important aspect of step c) is that the aqueous composition of the HMO is continuously or sequentially added to the drying but still wet agitated mixture, in the way it was disclosed in step b) of the method. Similarly, the water content of aqueous composition is evenly distributed throughout the drying but still wet agitated amorphous powder upon continuous agitation. It is important to set the addition rate of the aqueous composition corresponding to the drying capacity (water evaporation capacity) of the system (which is dependent of the rotation speed of the mixer, temperature, volume and optional vacuum) so that a dynamic equilibrium between the water/humidity introduced with the aqueous composition and the leaving water vapour is maintained. In this regard, the amount of water introduced by the addition of the aqueous composition is around the same than the amount of water removed in a given time window. For example, the addition rate of the aqueous composition is set in accordance with the condensed water which is continuously measured and monitored. Advantageously, the moisture/water content of the wet agitated powder is not more than 15%, for example not more than 12%, preferably not more than 10%, for example around 6-8% or 4-6%.

In one embodiment of the method, the dry agitated amorphous powder obtained in step a) is heated before the addition of the aqueous composition of the HMO according to step b). For instance, the dry powder is warmed to around 60-90° C., preferably 70-80° C.

In a preferred embodiment, the aqueous composition comprising an HMO to be added in step b) and/or in step c) is heated to a temperature of 40-90° C., preferably 60-80° C., and fed into the mixer at this temperature. Advantageously, the dry agitated amorphous powder obtained in step a) and/or the wet agitated powder mixture obtained in step b) and/or c) is/are also kept in the same temperature.

In one embodiment of the method, the dry powder fed into the mixer according to step a) is agitated at atmospheric pressure, and step b) and c) are performed under vacuum.

In one embodiment, steps a) and b) are performed at atmospheric pressure and step c) is conducted under vacuum.

In one embodiment of the method, the dry powder fed into the mixer according to step a) is agitated under vacuum, such as 20-100 mbars or 20-60 mbars.

In other embodiment, the dry powder fed into the mixer according to step a) is agitated under vacuum, such as 20-100 mbars or 20-60 mbars and the dry powder is warmed to around 60-90° C., preferably 70-80° C.

In other embodiment, step c) is operated under vacuum, preferably at 20-100 mbars or 20-60 mbars while the wet agitated powder mixture obtained in step c) is kept at around 60-90° C., preferably 70-80° C.

The method disclosed above including its preferred and more preferred embodiments leads to a dried amorphous powder of one or more HMOs that has a moisture content of below 10%, preferably below 8%, more preferably below 6%, for example below 4% down to 1-3%. According to one particular embodiment, the method disclosed above is performed in a vertical mixer dryer or a vertical vacuum mixer dryer. The vertical mixer dryer is equipped with a centrally positioned mixing arm with mixing blades, e.g. spiral mixing blades that allows efficient mixing and even distribution of the heat throughout the powder, therefore maximizing batch uniformity. The vertical mixer may be equipped with a cutting rotor that also helps to homogenize the agitated powder especially when agglomerates are locally formed.

The vertical mixer dryer, according to step a), is prefilled with the dry amorphous HMO powder. The amount of the dry amorphous powder is selected so that the aqueous composition can be conveniently sprayed or sprinkled in, on or to the dry powder and preferably the cutting rotor reached the powder bed.

Once the dry amorphous powder of HMO is placed into the mixer and agitated well, optionally at elevated temperatures and/or under vacuum as disclosed above, the aqueous composition of the HMO is sprayed or sprinkled in the form of fine droplets in, on or to the dry powder under constant agitation during step b) and the addition is continued throughout step c), preferably continuously. As the water vapour is continuously removed, the mass of the mixture of the agitated dry and wet powder is also continuously growing. Step c) is advantageously continued until the volume of the precipitated HMO powder reaches the maximum capacity of the mixer dryer. The addition of the aqueous solution is then terminated and the removal of the water is continued until the desired water content of the dried powder is obtained. In this regard, the operation of the method in a vertical mixer dryer represents a batch mode of drying.

A vertical mixer dryer equipped with a cutting rotor is suitable to obtain a desired particle size range besides drying and homogenizing the dried powder. The cutting rotor speed can be varied to control the particle size distribution.

According to another particular embodiment, the method disclosed above is performed in a horizontal contact dryer or a horizontal vacuum dryer. The horizontal dryer is equipped with a centrally positioned rotating paddle system that allows efficient mixing and even distribution of the heat throughout the powder, therefore maximizing batch uniformity. The horizontal contact dryer is designed so that feed inlet is situated in one of the ends and the dried product leaves the equipment at the other end.

The horizontal dryer, according to step a), is pre-filled, at least partially, with the dry amorphous HMO powder. The dry amorphous HMO powder may be a product from a previous run. Once the dry amorphous powder of HMO is placed into the horizontal dryer and agitated well, optionally at elevated temperatures and/or under vacuum as disclosed above, the aqueous composition, preferably solution, of the HMO is sprayed or sprinkled in the form of fine droplets in, on or to the dry powder under constant agitation during step b) and the addition is continued throughout step c). In parallel with the addition of the aqueous composition, the collection of the dried powder commences. The agitated wet powder, while it is being dried, is conveyed through the dryer be means of paddles located on a rotating shaft. The rotation speed of the shaft and/or the arrangement of the paddles on the shaft influences the residence time. In this regard, due to the movement of the agitated wet powder mixture from one end of the horizontal dryer towards the other end and the continuous water removal, the agitated wet powder mixture becomes dryer and dryer as a function of distance from the inlet. Consequently, the dried amorphous mixture leaves the dryer through the outlet at the other end. As long as the aqueous composition is added through the inlet in step c), the dried powder can be collected at the outlet. This way of operation of the method in a horizontal dryer represents a continuous mode of drying.

In one particular aspect, the one or more synthetic HMOs are produced by means of fermentation, prior to the drying step disclosed above.

Suitably, according to this aspect, one or more purification steps are carried out on the HMO fermentation broth, enzymatic reaction milieu or synthetic reaction mixture, prior to drying step disclosed above. The purification steps carried out on the fermentation broth, enzymatic reaction milieu or synthetic reaction mixture may be selected from one or more of:
removing solid material e.g. from the HMO synthesis milieu, such as fermentation material (such as proteins and DNA) or enzymatic reaction material (proteins);
removing salts and charged molecules (such as small DNA fragments, organic acids, peptides);
removing uncharged or non-charged material (e.g. lipids, polysaccharides e.g. endotoxin, colorants from fermentation);
removing HMO precursors and/or by-products;
separating HMOs, if an HMO mixture is produced;
removing an excess of water.

In aspects, said one or more synthetic HMOs are produced by means of fermentation and the fermentation broth is further processed to prepare an aqueous composition comprising said one or more synthetic human milk oligosaccharides (HMOs).

In one embodiment, the fermentation broth is treated by the following steps prior to drying:
A) clarifying the broth to remove suspended particulates and contaminants, particularly cells, cell components, insoluble metabolites and debris from a fermentation process; then
B) removing substantially all the proteins, as well as peptides, amino acids, RNA and DNA and any endotoxins and glycolipids that could interfere with the subsequent purification step, from the aqueous solution obtained in step a).

In step A), the broth is clarified in a conventional manner, e.g. by centrifugation or filtration. Preferably the aqueous medium is first flocculated and then centrifuged or filtered to remove any remaining insoluble particulates and contaminants, as well as cells and cell components and insoluble metabolites and debris.

In step B), proteins and related impurities are removed from the aqueous medium obtained previously in a conventional manner, e.g. by ultrafiltration, nanofiltration, tangential flow high-performance filtration, tangential flow ultrafiltration, affinity chromatography, ion exchange chromatography, hydrophobic interaction chromatography, gel filtration, size exclusion chromatography and/or active charcoal treatment. The active charcoal treatment helps to remove or at least reduce the amount of colorizing agents and/or water soluble contaminants, such as salts, if required. Ion exchange chromatography efficiently removes charged components such as salts, colour bodies, proteins, amino acids, lipids and DNAs.

Accordingly, the method my further comprise the following separation/purification steps of the fermentation broth in any order prior to drying:
i) ultrafiltration (UF),
ii) nanofiltration (NF), and
iii) treatment with an ion exchange resin.

Advantageously, step i) is conducted before step ii). More advantageously, the step i) is conducted before any of the steps ii) and iii). Preferably, the method is performed in the order where step ii) follows step i) and step iii) follows step ii). The method may further comprise an active charcoal treatment after UF, NF or ion exchange resin treatment.

The method provided herein may be supplemented by additional method steps, performed on the obtained solid amorphous HMO product, i.e. post-drying.

In one aspect, the methods described herein further comprise the step of milling the solid amorphous HMO product. Milling may provide a solid amorphous HMO powder with a various particle size distribution (D90) depending on the type of mill used e.g. impact mill, ball mill, forced sieve, or jet mill. As a further aspect, the methods may further comprise the step of sieving the solid amorphous HMO powder and separating the HMO powder into at least a first HMO powder fraction and a second HMO powder fraction. Sieving may provide HMO powder fractions with a particle size distribution (D90) depending the type of sieve used. The sieving or classification can be adapted to any customer requirement. From a food safety prospective a max. 1 mm sieving would be required, preferably 0.5-0.7 mm. However, if the mixer-dryer, e.g. the vertical mixer dryer is equipped with a cutting rotor or rotating knife, no separate milling step may be necessary to provide a solid amorphous HMO powder with the required particle size distribution (D90).

The solid HMO product may also be formulated in a food product, in particular a nutritional formulation, e.g. an infant nutritional formulation.

Nutritional formulations comprising the solid amorphous HMO product may be foods, drinks or feeds. The nutritional formulation may contain edible micronutrients, vitamins and minerals as well. The amounts of such ingredients may vary depending on whether the formulation is intended for use with normal, healthy infants, children, adults or subjects having specialized needs (e.g. suffering from metabolic disorders). Micronutrients include for example edible oils, fats or fatty acids (such as coconut oil, soy-bean oil, monoglycerides, diglycerides, palm olein, sunflower oil, fish oil, linoleic acid, linolenic acid etc.), carbohydrates (such as glucose, fructose, sucrose, maltodextrin, starch, hydrolysed cornstarch, etc.) and proteins from casein, soy-bean, whey or skim milk, or hydrolysates of these proteins, but protein from other source (either intact or hydrolysed) may be used as well. Vitamins may be chosen from the group consisting of vitamin A, B1, B2, B5, B6, B12, C, D, E, H, K, folic acid, inositol and nicotinic acid. The nutritional formulation may contain the following minerals and trace elements: Ca, P, K, Na, Cl, Mg, Mn, Fe, Cu, Zn, Se, Cr or I.

In a preferred embodiment the nutritional formulation is an infant nutritional formulation. Infant nutritional formulation means a foodstuff intended for particular nutritional use by infants during the first 4-6 months of life and satisfying by itself the nutritional requirements of infants. It may contain one or more probiotic Bifidobacterium species, prebiotics such as fructooligosaccharides and galactooligosaccharides, proteins from casein, soy-bean, whey or skim milk, carbohydrates such as lactose, saccharose, maltodextrin, starch or mixtures thereof, lipids (e.g. palm olein, sunflower oil, safflower oil) and vitamins and minerals essential in a daily diet. The infant formula contains solid HMO product in a total amount of 0.1-3.0 g/100 g formula.

In another preferred embodiment the nutritional formulation may be a food supplement comprising the solid amorphous HMO product. The food supplement may comprise one or more probiotics in an amount sufficient to achieve the desired effect in an individual, preferably in children and adults. The food supplement may also contain vitamins, minerals, trace elements and other micronutrients as well. The food supplement may be for example in the form of tablets, capsules, pastilles or a liquid. The supplement may contain conventional additives selected from but not limited to binders, coatings, emulsifiers, solubilising agents, encapsulating agents, film forming agents, adsorbents, carriers, fillers, dispersing agents, wetting agents, jellifying agents, gel forming agents, etc. The daily dose of solid HMO product ranges from 0.1 to 5.0 g.

The following numbered aspects of the invention are provided:
Aspect 1. A method for drying an aqueous composition of one or more human milk oligosaccharides (HMOs) and thereby providing a dried amorphous powder of said one or more HMOs, the method comprising:
   a) providing a dry amorphous powder comprising said one or more HMOs in a dryer suitable to agitate the dry amorphous powder mechanically, to obtain a dry agitated amorphous powder,
   b) feeding the aqueous composition of the one or more HMOs in, on or to the dry agitated amorphous powder to obtain a wet agitated mixture of the amorphous powder, and
   c) removing water from the wet agitated mixture of the amorphous powder at elevated temperature and/or under vacuum while the aqueous composition of the one or more HMOs is continuously or sequentially added to the wet agitated mixture of the amorphous powder.
Aspect 2. The method of aspect 1, wherein the dryer is designed to generate maximal possible dryer wall contact of the powder by mechanical mixing.
Aspect 3. The method of aspect 1 or 2, wherein the dryer is a vertical mixer dryer or a vertical vacuum mixer dryer.
Aspect 4. The method of aspect 3, wherein the vertical mixer dryer or a vertical vacuum mixer dryer is equipped with a centrally positioned mixing arm with spiral mixing blades.
Aspect 5. The method of aspect 4, wherein the vertical mixer dryer or a vertical vacuum mixer dryer is equipped with a cutting rotor.
Aspect 6. The method of aspect 1 or 2, wherein the dryer is a horizontal contact dryer or a horizontal vacuum dryer.
Aspect 7. The method of aspect 6, wherein the drying is performed continuously.
Aspect 8. The method of any one of the preceding aspects, wherein the aqueous composition of the one or more HMOs is an aqueous solution.
Aspect 9. The method of aspect 7, wherein the concentration of the HMO in the aqueous solution is at least 80%, preferably at least 90%, of its solubility concerning water.
Aspect 10. The method of any one of the preceding aspects, wherein the wet agitated mixture of the amorphous powder in step b) has a moisture content of not more than 15%.
Aspect 11. The method of aspect 10, wherein the wet agitated mixture of the amorphous powder in step b) has a moisture content of around 8-10%, 6-8% or 4-6%.
Aspect 12. The method of any one of the preceding aspects, wherein the temperature of the wet agitated mixture of the amorphous powder is kept at around 60-90° C.
Aspect 13. The method of aspect 12, wherein the temperature of the wet agitated mixture of the amorphous powder is kept at 70-80° C.
Aspect 14. The method of any one of the preceding aspects, wherein step c) is operated under vacuum.
Aspect 15. The method of aspect 14, wherein the vacuum is 20-100 mbars, preferably 20-60 mbars.
Aspect 16. The method of any one of the preceding aspects, wherein the wet agitated mixture of the amorphous powder in step c) has a moisture content of not more than 15%.
Aspect 17. The method of aspect 16, wherein the wet agitated mixture of the amorphous powder in step c) has a moisture content of around 8-10%, 6-8% or 4-6%.
Aspect 18. The method of any one of the preceding aspects, wherein in step c) the amount of water introduced by the addition of the aqueous composition is around the same than the amount of water removed.
Aspect 19. The method of any one of the preceding aspects, wherein the dry agitated amorphous powder obtained in step a) is warmed to around 60-90° C., preferably 70-80° C.

Aspect 20. The method of any one of the preceding aspects, wherein the aqueous composition, preferably the aqueous solution to be added in step b) and/or in step c) is pre-heated to a temperature of 40-90° C., preferably 60-80° C., before addition.
Aspect 21. The method of any one of the preceding aspects, wherein the dry powder fed into the mixer according to step a) is agitated at atmospheric pressure, and step b) and c) are performed under vacuum.
Aspect 22. The method of any one of the preceding aspects, wherein step c) is operated under vacuum, preferably at 20-100 mbars or 20-60 mbars, while the wet agitated powder mixture is kept at around 60-90° C., preferably 70-80° C.
Aspect 23. The method of any one of the preceding aspects, wherein the dried amorphous powder of said one or more HMOs obtained has a moisture content of below 8%, preferably below 6%, for example below 4% down to 1-3%.
Aspect 24. The method of any one of the preceding aspects, wherein the HMO is a neutral HMO.
Aspect 25. The method of aspect 24, wherein the neutral HMO is a non-fucosylated HMO.
Aspect 26. The method of aspect 25, wherein the non-fucosylated HMO is LNT or LNnT.
Aspect 27. The method of aspect 24, wherein the neutral HMO is a fucosylated HMO.
Aspect 28. The method of aspect 27, wherein the fucosylated HMO is 2′-FL and/or DFL.
Aspect 29. The method of any one of the preceding aspects, wherein the HMO is a sialylated HMO.
Aspect 30. The method of aspect 29, wherein the sialylated HMO is 3′-SL or 6′-SL.
Aspect 31. The method of any one of the preceding aspects, wherein the HMO is produced by fermentation.
Aspect 32. A dried amorphous powder of one or more HMOs obtained or obtainable by a method defined in any one of the aspects 1 to 31.
Aspect 33. The dried amorphous powder of one or more HMOs of aspect 32 having a moisture content of below 8%, preferably below 6%, for example below 4% down to 1-3%.
Aspect 34. A nutritional formulation comprising the dried amorphous powder of one or more HMOs according to aspect 32 or 33.
Aspect 35. The nutritional formulation of aspect 34, which is a food, drink, feed, infant formula, medical food or food supplement.

The technology has been described with reference to a number of aspects and embodiments. These aspects and embodiments can be combined as desired by the skilled person, while remaining within the scope of the present invention, as defined by the appended claims. In particular, features relating to the methods described herein are also applicable to the solid HMO products, inasmuch as the method features give rise to identifiable differences in the solid HMO products themselves.

### EXAMPLES

### Example 1

Vertical vacuum mixer dryer VMT 200-1730 (amixon®) equipped with S-type blades and a horizontal cutting rotor was filled with 70 kg of amorphous 2′-FL. The equipment was heated (thermal oil in the jacket) to 130° C. (inlet temperature) gradually. The central mixer was set to 50 rpm. When the temperature of the agitated powder reached 65° C. (inlet temperature 125° C.), the cutting rotor was set to 1500 rpm and vacuum was applied. At the pressure of 220 mbar (powder temperature 74° C.), the addition of a preheated (80° C.) solution of 2′-FL (Brix 56) started through a heated liquid addition hose (8 mm Ø, standard lance with Spraying systems nozzle size 10) at an air pressure of 2 bars with 500 g solution per minute flow rate. The flow rate was raised to 800 g/min, then 1050 g/min while the internal pressure was maintained 90-110 mbars and the temperature of the powder was 71-77° C. 50 kg of 2′-FL solution was injected during one hour. A vacuum of 50-80 mbars was maintained for additional 10-15 minutes. The process was stopped and 93 kg of dried 2′-FL powder was collected which was proved to be amorphous by its X-ray powder diffraction analysis. The residual moisture of the final powder was 2.1% (moisture analyser). There were no sticky residues found in the mixing chamber.

1. A method for drying an aqueous composition of one or more human milk oligosaccharides (HMOs) and thereby providing a dried amorphous powder of said one or more HMOs, the method comprising:
a) providing a dry amorphous powder comprising said one or more HMOs in a dryer suitable to agitate the dry amorphous powder mechanically, to obtain a dry agitated amorphous powder, preferably the dryer is designed to generate maximal possible dryer wall contact of the powder by mechanical mixing,
b) feeding the aqueous composition of the one or more HMOs in, on or to the dry agitated amorphous powder to obtain a wet agitated mixture of the amorphous powder, and
c) removing water from the wet agitated mixture of the amorphous powder at elevated temperature and/or under vacuum while the aqueous composition of the one or more HMOs is continuously or sequentially added to the wet agitated mixture of the amorphous powder.

2. The method according to claim 1, wherein the dryer is a vertical mixer dryer or a vertical vacuum mixer dryer.

3. The method according to claim 1, wherein the dryer is a horizontal contact dryer or a horizontal vacuum dryer.

4. The method according to claim 1, wherein the aqueous composition of the one or more HMOs is an aqueous solution.

5. The method according to claim 4, wherein the concentration of the HMO in the aqueous solution is at least 80% of its solubility concerning water.

6. The method according to claim 1, wherein the wet agitated mixture of the amorphous powder in step b) and step c) has a moisture content of not more than 15%.

7. The method according claim 1, wherein the temperature of the wet agitated mixture of the amorphous powder is kept at around 60-90° C., preferably at 70-80° C.

8. The method according to claim 1, wherein step c) is operated under vacuum.

9. The method according to claim 8, wherein the wet agitated powder mixture is kept at around 60-90° C..

10. The method according to claim 1, wherein the one or more HMOs comprises a neutral HMO.

11. The method according to claim 1, wherein the one or more HMOs is comprises a sialylated HMO.

12. The method according to claim 1, wherein the one or more HMOs is 2′-FL, 3-FL, DFL, LNT, LNnT, LNFP-I, 3′-SL, 6′-SL, or any mixture thereof.

13. The method according to claim 1, wherein the one or more HMOs is produced by fermentation.

14. A dried amorphous powder of one or more HMOs obtained or obtainable by a method defined in claim 1, wherein the dried amorphous powder of one or more HMOs has a moisture content of below 8%.

15. A nutritional formulation comprising the dried amorphous powder of one or more HMOs according to claim 14.

16. The nutritional formulation of claim 15, wherein the nutritional formulation is a food, drink, feed, infant formula, medical food, or food supplement.

17. The dried amorphous powder of claim 14, wherein the the dried amorphous powder of one or more HMOs has a moisture content of below 4%.

18. The method according to claim 6, wherein the moisture content ranges from around 4% to around 10%.

19. The method according to claim 8, wherein the vacuum is 20-60 mbars.

20. The method according to claim 9, wherein the wet agitated powder mixture is kept at 70-80° C.
